(19) United States
(12) Patent Application Publication
(10) Pub. No.: US 2016/0206579 A1
(43) Pub. Date: Jul. 21, 2016

(54) TAURINE OR TAURINE-LIKE SUBSTANCES FOR THE PREVENTION AND TREATMENT OF A DISEASE ASSOCIATED WITH RETINAL GANGLION CELL DEGENERATION

(71) Applicant: Serge PICAUD, Paris (FR)

(72) Inventor: Serge PICAUD, Paris (FR)

(73) Assignee: INSERM (Institut National de la Santé et de la Recherche Médicale), Paris (FR)

(21) Appl. No.: 15/064,151

(22) Filed: Mar. 8, 2016

Related U.S. Application Data
(63) Continuation of application No. 13/190,812, filed on Jul. 26, 2011.
Application No. 13/190,812 is a continuation-in-part of application No. PCT/EP2010/051384, filed on Feb. 4, 2010.

(30) Foreign Application Priority Data
Feb. 4, 2009 (EP) 09305105.0

(51) Int. Cl. A61K 31/185; A61K 45/06

(52) U.S. Cl. CPC A61K 31/185; A61K 45/06

(57) ABSTRACT

The present invention relates to taurine or taurine-like substances for the prevention and treatment of a disease associated with retinal ganglion cell degeneration. More particularly the invention relates to a substance selected from the group consisting of taurine, a taurine precursor, a taurine metabolite, a taurine derivative, a taurine analog and a substance required for the taurine biosynthesis for the prevention and treatment of a disease associated with retinal ganglion cell degeneration.

This application claims benefit of and is a continuation-in-part (CIP) of International patent application PCT/EP2010/051384, filed 4 Feb. 2010, and of European patent application EP 09305105.0, filed 4 Feb. 2009, the complete contents of both of which are hereby incorporated by reference.

### FIELD OF THE INVENTION

The present invention relates to taurine or taurine-like substances for the prevention and treatment of a disease associated with retinal ganglion cell (RGC) degeneration.

### BACKGROUND OF THE INVENTION

Glaucoma denotes a group of diseases of the optic nerve involving loss of retinal ganglion cells in a characteristic pattern of optic neuropathy. Raised intraocular pressure is a significant risk factor for developing glaucoma (above 22 mmHg). Untreated glaucoma leads to permanent damage of the optic nerve and resultant visual field loss, which can progress to blindness. Glaucoma can be divided roughly into two main categories, “open angle” or chronic glaucoma and “closed angle” or acute glaucoma. Angle closure, acute glaucoma appears suddenly and often with painful side effects and so is usually diagnosed quickly, although damage and loss of vision can also occur very suddenly. Open angle, chronic glaucoma tends to progress more slowly and so the patient may not notice it until the disease has progressed quite significantly. Glaucoma has been nicknamed the “sneak thief of sight” because the loss of visual field often occurs gradually over a long time and may only be recognized when it is already quite advanced. Once lost, this damaged visual field can never be recovered. Worldwide, it is the second leading cause of blindness. Glaucoma affects one in two hundred people aged fifty and younger, and one in ten over the age of eighty.

Thus, there is a need in the art for substances that would allow preventing and treating glaucoma.

The nature of the mechanistic link between high intraocular pressure and loss of retinal ganglion cells is not firmly established. Although less direct insults have occasionally been suggested, trauma at the optic nerve head, the location where the axons of the ganglion cells join together to leave the globe, has been a leading possibility. Generally speaking, this could occur by compression or by pressure on the axons at their point of exit, but the exact pathophysiological events remain unknown (Quigley H A, 1987; Quigley H A, 1999; Libby R T et al., 2005; Whitmore A V et al., 2005).

Prevention of retinal ganglion cell (RGC) degeneration may also be useful for the treatment of other forms of optic nerve atrophy like the Leber hereditary optic neuropathy or pathologies with retinal ischemia like vascular occlusions.

### SUMMARY OF THE INVENTION

The present invention relates to a substance selected from the group consisting of taurine, a taurine precursor, a taurine metabolite, a taurine derivative, a taurine analog and a substance required for the taurine biosynthesis for the prevention and treatment of a disease associated with retinal ganglion cell degeneration.

The present invention also relates to a pharmaceutical composition for the prevention and treatment of a disease associated with retinal ganglion cell degeneration which comprises a substance as above described and optionally one or more pharmaceutically acceptable excipients.

### DETAILED DESCRIPTION OF THE INVENTION

The inventors have demonstrated that taurine prevents retinal ganglion cells degeneration, and therefore may be useful for the prevention and treatment of a disease associated with retinal ganglion cell degeneration.

Thus, an object of the present invention relates to a substance selected from the group consisting of taurine, a taurine precursor, a taurine metabolite, a taurine derivative, a taurine analog and a substance required for the taurine biosynthesis, for the prevention and treatment of a disease associated with retinal ganglion cell degeneration.

In a particular embodiment, diseases associated with retinal ganglion cell degeneration include but are not limited to glaucoma and other forms of optic nerve atrophy like the Leber hereditary optic neuropathy or pathologies with retinal ischemia like vascular occlusions.

In another embodiment, diseases associated with retinal ganglion cell degeneration also include but are not limited to arteritic ischemic optic neuropathy (giant cell arteritis), nonarteritic ischemic optic neuropathy, infiltrative optic neuropathy (sarcoidosis), infectious optic neuropathy (syphilis, lyme, toxoplasmosis, herpes zoster), optic neuritis from demyelinating disease, posradiation optic neuropathy, acrodermatitis enteropathica, hereditary optic neuropathy (Leber's hereditary optic neuropathy, dominant optic neuropathy), compressive optic neuropathy (orbital pseudotumor, thyroid eye disease), autoimmune optic neuropathy (Lupus).

In another embodiment, the substance according to the invention may be useful for the treatment of cholestatic liver disease, nutritional optic neuropathy, ketogenic diet, thiamine deficiency.

As used herein, the term “taurine” refers to 2-aminoethanesulfonic acid.

As used herein, “taurine precursors” encompass substances that, when they are administered to a human or an animal, can be transformed, directly or indirectly, into taurine.

As used herein, “taurine metabolites” encompass substances that are produced in vivo by transformation of taurine.

As used herein, “taurine derivatives” encompass substances that are structurally close to taurine but possess at least one structural difference, such as one or more chemical changes, e.g. at least one replacement of an atom or a chemical group found in taurine by a distinct atom or a distinct chemical group.

As used herein, “taurine analogs” encompass substances that are chemically distinct from taurine but which exert the same biological activity.

As used herein, “substances required for taurine biosynthesis” encompass all substances that are involved in the in vivo taurine biosynthesis including enzymes and enzyme cofactors, thus including cysteine dioxygenase (EC 1.13.11), sulfinoalanine decarboxylase (EC 4.1.1.29) and cofactors thereof.

As intended herein, taurine precursors, taurine metabolites, taurine derivatives, taurine analogs and substances required for the taurine biosynthesis may be collectively termed “taurine-like substances”.

In a particular embodiment, taurine precursors are selected from the group consisting of cysteine, cystathionine, homocysteine, S-adenosylhomocysteine, serine, N-acetyl-cysteine, glutathione, N-formylmethionine, S-adenosylmethionine, betaine and methionine.

In another particular embodiment, taurine metabolites are selected from the group consisting of hypotaurine, thiotaurine, taurocholate, tauret also known as retinyliden taurine ((3,7-dimethyl-9-(2,6,6-trimethyl-1-cyclohexen-1-yl)-2,4,6,8-nonatetraen-1-imido-(N-ethane sulfonic acid)).

In another particular embodiment, taurine derivatives are selected from different entities including the group consisting of acetylhomotaurinate, and piperidino-, benzamido-, phthalimido- or phenylsuccinylimido taurine derivatives. Such taurine derivatives are described notably by Kontro et al. (1983) and by Andersen et al. (1984). Derivatives include for instance taurolidine (4,4′-methylene-bis(tetrahydro-2H-1,2,4-thiadiazine-1,1-dioxide or taurolin), taurultam and taurinamide, chlorohydrate-N-isopropylamide-2-(1-phenylethyl)amino ethanesulfonic acid.

In another particular embodiment taurine analogs are selected from the group consisting of (+/−) piperidine-3-sulfonic acid (PSA), 2-aminoethylphosphonic acid (AEP), (+/−) 2-acetylaminocyclohexane sulfonic acid (ATAHS), 2-aminobenzenesulfonate (ANSA), hypotaurine, ±trans-2-aminocyclopentanesulfonic acid (TAPS) 8-tétrahydroquinoléine sulfonic acid (THQS), N-2-hydroxyethylpiperazine-N′-2-ethane sulphonic acid (HEPES), beta-alanine, glycine, guanidinoethylsulfate (GES), 3-acétamido-1-propanesulfonic acid (acamprosate).

In another particular embodiment, substances required for taurine biosynthesis are selected from the group consisting of vitamin B6 (or pyridoxal-5′-phosphate), vitamin B12 (cobalamin), folic acid, riboflavin, pyridoxine, niacin, thiamine (thiamine pyrophosphate) and pantothenic acid.

In a particular embodiment the substance selected from the group consisting of taurine, a taurine precursor, a taurine metabolite, a taurine derivative, a taurine analog and a substance required for the taurine biosynthesis is used without:
A cyclic GMP increasing agent comprising folic acid,
A cell membrane integrity maintenance agent comprising D,α-tocopherol and ascorbate, and
A hyperinsulinemia modulating agent comprising α-lipoic acid.

As used herein, the term “a cyclic GMP increasing agent” denotes a compound which increases the concentration of cGMP. This results in increased blood flow, increased endothelial cell proliferation, reduced endothelial permeability, inhibited vascular smooth muscle proliferation and a lowered rate of both neural and glial apoptosis.

As used herein, the term “a cell membrane integrity maintenance agent” denotes a compound which is able to keep a membrane with selective permeability and selective active-transfer mechanisms.

As used herein, the term “a hyperinsulinemia modulating agent” denotes a compound which is able to limit the excess levels of circulating insulin in the blood.

In another preferred embodiment, the substance selected from the group consisting of taurine, a taurine precursor, a taurine metabolite, a taurine derivative, a taurine analog and a substance required for the taurine biosynthesis, is used alone.

In another embodiment, the substance according to the invention may be useful for preventing the retinal ganglion cell degeneration induced by antimicrobial or anti-malaria drug such as chloramphenicol, chloroquine, clioquinol, dapsone, ethambutol, iodochlorohydroxyquinoline, isoniazide, linezolid, streptomycin.

In another particular embodiment, the invention relates to an antimicrobial or anti-malaria composition comprising a substance according to the invention and at least one active ingredient selected from the group consisting of chloramphenicol, chloroquine, clioquinol, dapsone, ethambutol, iodochlorohydroxyquinoline, isoniazide, linezolid, streptomycin.

In another embodiment, the substance according to the invention may be useful for preventing the retinal ganglion cell degeneration induced by an immunomodulator or immunosuppressive drug such as cyclosporine, interferon-alpha, tacrolimus (FK506).

In another particular embodiment, the invention relates to an immunomodulator or immunosuppressive composition comprising a substance according to the invention and at least one active ingredient selected from the group consisting of cyclosporine, interferon-alpha, tacrolimus (FK506).

In another embodiment, the substance according to the invention may be useful for preventing the retinal ganglion cell degeneration induced by a chemotherapeutics drug such as carboplatin, chlorambucil, cisplatin, 5-fluorouracil, methotrexate, nitrosureas (BCNU, CCNU, ACNU), paclitaxel, tamoxifen, 5-vincristine, cytosine arabinoside, purine analogues, procarbazine, cyclophosphamide, vinca alkaloids.

In another particular embodiment, the invention relates to a chemotherapeutic composition comprising a substance according to the invention and at least one active ingredient selected from the group consisting of carboplatin, chlorambucil, cisplatin, 5-fluorouracil, methotrexate, nitrosureas (BCNU, CCNU, ACNU), paclitaxel, tamoxifen, 5-vincristine, cytosine arabinoside, purine analogues, procarbazine, cyclophosphamide, vinca alkaloids.

In another embodiment, the substance according to the invention may be useful for preventing the retinal ganglion cell degeneration induced by a drug such as amiodarone, amantidine amoproxen, cafergot, chlorpropamide, cimetidine, clomiphene citrate, deferoxamine, disulfiram, emetine, infliximab, pheniprazine, quinine, PDE inhibitors (sildenafil, tadalafil, vardenafil), bendroflumethiazide, chorothiazide, chlortalidone, hydrochlorothiazide, hydroflumethiazide, indapamide, methyclothiazide, metolazone, polythiazide, trichlomethiazide.

In another particular embodiment, the invention relates to a therapeutic composition comprising a substance according to the invention and at least one active ingredient selected from the group consisting of amiodarone, amantidine amoproxen, cafergot, chlorpropamide, cimetidine, clomiphene citrate, deferoxamine, disulfiram, emetine, infliximab, pheniprazine, quinine, PDE inhibitors (sildenafil, tadalafil, vardenafil), bendroflumethiazide, chorothiazide, chlortalidone, hydrochlorothiazide, hydroflumethiazide, indapamide, methyclothiazide, metolazone, polythiazide, trichlormethiazide.

In another embodiment, the substance according to the invention may be useful for preventing the toxicity induced by a molecule such as alcohol, arsacetin, caron monoxide, carbon disulfide, carbon tetrachloride, cobalt chloride, ethchorvynol, ethylene glycol, hexachlorophene, iodoform, lead, mercury, methanol, methyl acetate, methyl bromide, octamoxin, organic solvents, perchloroethylene, pheniprazine, plasmocid, styrene, thallium, trichloroethylene, triethyl tin, tobacco, toluene.

In another preferred embodiment, the substance according to the invention may be useful for the treatment of a disease associated with photoreceptors degeneration.

In another particular embodiment, the substance according to the invention may be useful for enhance the survival of retinal ganglion cells in a disease associated with photoreceptors degeneration.

This invention also relates to a therapeutic method for the prevention or treatment of a disease associated with retinal ganglion cell degeneration, wherein said method comprises a step of administering to a subject in need thereof with an effective amount of a substance selected from the group consisting of taurine, a taurine precursor, a taurine metabolite, a taurine derivative, a taurine analog and a substance required for the taurine biosynthesis.

According to the invention, the term “subject” or “patient” and “subject in need thereof” or “patient in need thereof”, is intended for a human or a non-human mammal.

Generally speaking, a “therapeutically effective amount”, or “effective amount”, or “therapeutically effective”, as used herein, refers to that amount which provides a therapeutic effect for a given condition and administration regimen. This is a predetermined quantity of active material calculated to produce a desired therapeutic effect in association with the required additive and diluent; i.e., a carrier, or administration vehicle. Further, it is intended to mean an amount sufficient to reduce and most preferably prevent a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluents; i.e., carrier, or additive.

The present invention also pertains to pharmaceutical compositions comprising a substance selected from the group consisting of taurine, a taurine precursor, a taurine metabolite, a taurine derivative, a taurine analog and a substance required for the taurine biosynthesis, for the prevention and treatment of a disease associated with retinal ganglion cell degeneration. In a pharmaceutical composition according to the invention, the amount of the taurine or a taurine-like substance, is adapted so that the said pharmaceutical composition is adapted so that the dosage form used allows the administration of an amount of taurine or of the taurine-like substance ranging from 10 μg to 10 grams per day for a human adult patient having a mean weight of 80 kilos.

Indeed, in a pharmaceutical composition, the active ingredient is used in combination with one or more pharmaceutically or physiologically acceptable excipients.

Generally, a pharmaceutical composition according to the invention comprises an amount of excipient(s) that ranges from 0.1% to 99.9% by weight, and usually from 10% to 99% by weight, based on the total weight of the said pharmaceutical composition.

By “physiologically acceptable excipient or carrier” is meant solid or liquid filler, diluents or substance which may be safely used in systemic or topical administration. Depending on the particular route of administration, a variety of pharmaceutically acceptable carriers well known in the art include solid or liquid fillers, diluents, hydrotropes, surface active agents, and encapsulating substances.

Pharmaceutically acceptable carriers for systemic administration that may be incorporated in the composition of the invention include sugar, starches, cellulose, vegetable oils, buffers, polyols and alginic acid. Specific pharmaceutically acceptable carriers are described in the following documents, all incorporated herein by reference: U.S. Pat. No. 4,401,663, Buckwalter et al. issued Aug. 30, 1983; European Patent Application No. 089710, LaHann et al. published Sep. 28, 1983; and European Patent Application No. 0068592, Buckwalter et al. published Jan. 5, 1983. Preferred carriers for parenteral administration include propylene glycol, pyrrolidone, ethyl oleate, aqueous ethanol, and combinations thereof.

Representative carriers include acacia, agar, alginates, hydroxyalkylcellulose, hydroxypropyl methylcellulose, carboxymethylcellulose, carboxymethylcellulose sodium, carrageenan, powdered cellulose, guar gum, cholesterol, gelatin, gum agar, gum arabic, gum karaya, gum ghatti, locust bean gum, octoxynol 9, oleyl alcohol, pectin, poly(acrylic acid) and its homologs, polyethylene glycol, polyvinyl alcohol, polyacrylamide, sodium lauryl sulfate, poly(ethylene oxide), polyvinylpyrrolidone, glycol monostearate, propylene glycol monostearate, xanthan gum, tragacanth, sorbitan esters, stearyl alcohol, starch and its modifications. Suitable ranges vary from about 0.5% to about 1%.

For formulating a pharmaceutical composition according to the invention, the one skilled in the art will advantageously refer to the last edition of the European pharmacopoeia or of the United States pharmacopoeia.

Preferably, the one skilled in the art will refer to the fifth edition “2005” of the European Pharmacopoeia, or also to the edition USP 28-NF23 of the United States Pharmacopoeia.

Pharmaceutical composition according to the invention may also contain other compounds, which may be biologically active or inactive. For example, substance according to the invention may be combined with another agent, in a treatment combination, and administered according to a treatment regimen of the present invention. Such combinations may be administered as separate compositions, combined for delivery in a complementary delivery system, or formulated in a combined composition, such as a mixture or a fusion compound.

The pharmaceutical composition of the invention may be formulated for a topical, oral, intranasal, parenteral, intraocular, intravenous, intramuscular or subcutaneous or eye drop administration and the like.

In another preferred embodiment, the pharmaceutical composition of the invention is useful for the treatment of a disease associated with photoreceptors degeneration.

In another particular embodiment, the pharmaceutical composition of the invention is useful for enhance the survival of retinal ganglion cells in a disease associated with photoreceptors degeneration.

In another particular embodiment, the invention relates to a pharmaceutical composition comprising a substance according to the invention and at least one active ingredient selected from the group consisting latanoprost, timolol, travoprost, dorzolamide, brimonidine, bimatoprost, apraclonidine, dipivephrine, propine, acetazomide, brinzolamide.

In another preferred embodiment, the pharmaceutical composition comprising a substance according to the invention and at least one active ingredient selected from the group consisting latanoprost, timolol, travoprost, dorzolamide, brimonidine, bimatoprost, apraclonidine, dipivephrine, propine, acetazomide, brinzolamide, is useful for the treatment of glaucoma.

In another embodiment, the pharmaceutical composition comprising a substance according to the invention and at least one active ingredient selected from the group consisting latanoprost, timolol, travoprost, dorzolamide, brimonidine, bimatoprost, apraclonidine, dipivephrine, propine, acetazomide, brinzolamide may be formulated for eye drop administration.

The invention will be further illustrated by the following figures and examples. However, these examples and figures should not be interpreted in any way as limiting the scope of the present invention.

### FIGURES

FIG. 1: Effect of Vigabatrin with or without taurine supplementation on retinal ganglion cell (RGC) survival. Quantification of Brn3A-positive retinal ganglion cells in retinal sections of control animals (Cont), vigabatrin-treated rats (VGB) and vigabatrin-treated rats supplemented with taurine (VGB+taurine). The differences between control animals and the two other groups were statistically significant and the difference between the two groups of VGB-treated animals with or without taurine supplementation was also significant. (*p<0.001, ° p<0.005, s.e.m. n=10).

FIG. 2: Protective effect of taurine on the survival of pure cultured retinal ganglion cells (RGCs). Histogram showing the quantification of RGC survival at 6 days in vitro (6 DIV) either in control condition (negative control) or in presence of 1 mM of taurine; addition of the B27 supplement to the culture medium was taken as a positive control. Data are expressed as a percentage of RGC survival at 6 DIV with respect to that at 1 day in vitro (1 DIV). They are provided as mean±SEM of 24 independent experiments. Statistical significances were calculated with respect to the control group (*P<0.05 and ***P<0.001, One-Way ANOVA, followed by a bonferroni post-hoc test).

FIG. 3A-E: Protective action of taurine treatment against RGC degeneration in Long-Evans rats following an episcleral vein occlusion, an induced animal model of glaucoma. A: Taurine plasma levels in long-Evans rats without (water) or with taurine (0.2M) addition in their drinking for 3 months. Each bar represents the taurine plasma level in μmol/L. Data are the means±SEM obtained from 8 animals for each group. **P<0.01 as compared to the water group (student t test).
B: IOP levels measured at regular time intervals on the operated and unoperated eyes after the episcleral vein occlusion (operation) in control (water) and taurine-treated animals as in (A). Data are the means±SEM obtained from 24 animals. ***P<0.001 as compared to the respective control eye of either taurine treated or control rats (one-way ANOVA followed by a bonferroni post-hoc test).
C: Photopic ERG recorded from operated and unoperated eyes after 3 months of episcleral vein occlusion in taurine-treated rats and control (water) rats.
D: Photopic ERG amplitudes measured from the unoperated and operated eyes in taurine-treated rats (n=24) or in control (water) rats (n=40) after 3 months. Data (μVolt) are means±SEM. *P<0.05 and ***P<0.001 as compared to the indicated groups (One way ANOVA followed to a bonferroni post-test).
E: Quantification of the RCG densities on retinal sections from operated eyes in taurine treated and control (water) rats (n=4). RGC were immunostained using Brn-3a specific marker. Data, expressed as number of RGC per mm of retinal section, are means±SEM. **P<0.01 as compared to the control rats (One way ANOVA followed to a bonferroni post-test).

FIG. 4A-C: Protective action of taurine treatment against RGC degeneration in DBA/2J mice, a natural animal model of pigmentary glaucoma. A: Taurine plasma levels in DBA/2J mice without (water) or with taurine supplementation (0.2M) in their drinking water from 8 to 12 months of age. Data are the means±SEM obtained from 10 animals per group. **P<0.01 as compared to control group (student t test).
B: Photopic ERG recorded in 12-month old DBA/2J mice without (water) or with taurine supplementation for 4 months.
C: Photopic ERG amplitudes measured in the taurine-treated mice (n=5) or in control (water) DBA/2J mice (n=3). ***P<0.001 as compared to the indicated groups (One way ANOVA followed to a bonferroni post-test).

FIG. 5: Protective action of taurine treatment against RGC degeneration in P23H rats, an animal model of retinitis pigmentosa. Quantification of brn3a immunopositive RGC in retinal sections from Sprague-Dawley control animals (CTL, black bar), from untreated heterozygous P23H rats (light gray bar) and from taurine treated P23H rats (dark dotted gray bar). The average RGC number was 411±19 in CTL Sprague-Dawley rats (n=7), 298±_12 in untreated heterozygous P23H rats (n=7) and 344±8 in taurine treated P23H rats (n=6). Difference between control animals and untreated heterozygous P23H rats: P<0.001. Difference between untreated heterozygous P23H and taurine treated heterozygous P23H: P<0.01 (One way ANOVA followed by Newman-Keuls Multiple Comparison Test). Data are the means±SEM.

FIG. 6A-E: Taurine prevents the RGC death in NMDA-treated retinal explants.
A) Digitalized reconstruction of a whole flat-mounted retinal explant immunolabeled with the Brn-3a antibody. B-D) Representative enlarged fields from flat-mounted retinal explants acquired with the automated platform, showing Brn-3a-immunopositive RGCs in a control untreated condition (Cont; B) after NMDA application (100 μM NMDA, C) or after co-application of NMDA plus taurine (1 mM) (NMDA+Taur; D) for 4 days. E) Quantification of RGC densities from whole flat-mounted retinal explants using the automated counting platform. Data are expressed as densities of Brn-3a-immunopositive RGCs (cell/mm², means s.e.m.; control group: n=33; NMDA group: n=31; NMDA+Taur group: n=23, ***p<0.001, *p<0.05; One-way ANOVA followed by a Bonferroni post-hoc test). The scale bar represents 100 μm.

### EXAMPLE 1

### Effects of Taurine in a Model of Retinal Ganglion Cell Degeneration (RGC) Induced by Vigabatrin

Material & Methods:

Animal Treatments:

Breeds of Wistar rats Rj Wi IOPS Han were purchased from Janvier (Le Genest-St-Isle, France). Vigabatrin (VGB) dissolved in 0.9% NaCl was administered at 0.6 mg/jour (6 mg/ml, 0.1 ml) to rats by daily intraperitoneal injection for 25 days from age 4 days. These daily doses (50 mg/kg for rats of 12 g) are in-line with those described for the treatment of epilepsy (adult patients: 1-6 mg/kg; children:50-75 mg/kg; or infants: 100-150 mg/kg) (Aicardi et al., 1996; Chiron et al., 1997; Lux et al., 2004). Taurine supplementation was administered by intraperitoneal injections at a concentration of 5 mg/day (50 mg/ml in 0.1 ml).

Histology:

Animals were anesthetized by intraperitoneal injection (0.8 to 1.2 ml/kg) of a solution containing ketamine (40 mg/ml) and xylazine (4 mg/ml Rompum). They were then perfused first with heparin 1000 UI/ml in saline solution (0.9% NaCl), then with fixative containing 4% Paraformaldehyde (Merck chemicals ref.: 100496) and 0.2% glutaraldehyde (fluka 25%). Eye cups were isolated and fixed overnight at 4° C. in 4% (wt/vol) paraformaldehyde in phosphate buffered saline (PBS; 0.01M, pH 7.4). The tissue was cryoprotected in successive solutions of PBS containing 10%, 20% and 30% sucrose at 4° C., oriented along the dorso-ventral axis and embedded in OCT (Labonord, Villeneuve d'Ascq, France). Retinal sections (8-10 μm thickness) were permeabilised for five minutes in PBS containing 0.1% Triton X-100 (Sigma, St. Louis, Mo.), rinsed, and incubated in PBS containing 1% bovine serum albumin (Eurobio, Les-Ulis, France), 0.1% Tween 20 (Sigma), and 0.1% sodium azide (Merck, Fontenay-Sous-Bois, France) for two hours at room temperature. The primary Brn3A monoclonal antibody (Chemicon) was added to the solution and incubated for two hours at room temperature. Sections were rinsed and then incubated with the secondary antibody, rabbit anti-mouse IgG conjugated to either Alexa™594 (1:500, Molecular Probes) for two hours. Sections were rinsed, mounted with Fluorsave reagent (Calbiochem) and viewed with a Leica microscope (LEICA DM 5000B) equipped with a Ropper scientific camera (Photometrics cool SNAP™ FX). Quantifications were achieved on whole vertical sections cut along the dorso-ventral axis and crossing the optic nerve.

Statistical Analysis:

Statistical analysis of the results was performed by a one-way analysis of variance with the Student-Newman Keuls test (Sigmastat).

Results:

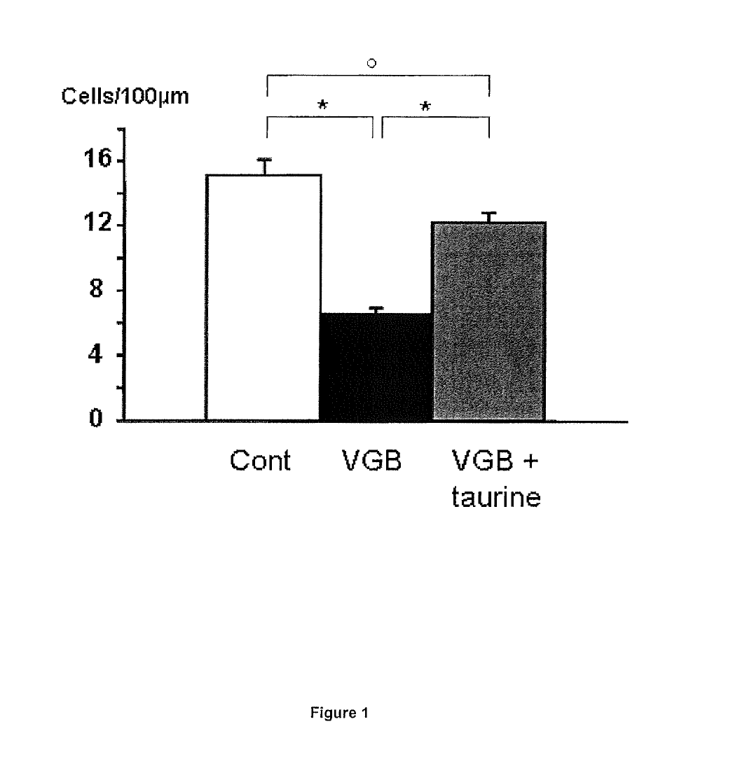

It is well documented that vigabatrin (VGB) induced an optic nerve atrophy in treated infants (Frisen and Malmgren, 2003; Viestenz et al., 2003; Buncic et al., 2004). The inventors have demonstrated that when young rats were treated with VGB, the number of retinal ganglion cells was decreased by 56% (control: 15.06±0.93; VGB: 6.56±0.32, s.e.m., n=10). The difference was statistically significant (p<0.001) (FIG. 1). When VGB-treated rats received taurine supplementation, although the number of retinal ganglion cells remained decreased with respect to control (VGB+taurine: 12, 13±0.62, s.e.m., n=10), it was 84.9% greater than in the VGB-treated group without supplementation. The difference with the group of VGB-treated rats was statistically significant (p<0.001). However the taurine supplementation did not completely rescue retinal ganglion cells, the difference with the control group remained significant (p<0.005) (FIG. 1). These results indicated that taurine supplementation can prevent retinal ganglion cell degeneration.

### EXAMPLE 2

### Protective Effect of Taurine on the Survival of Pure Cultured Retinal Ganglion Cells (RGCS)

Material & Methods:

Primary cultures of pure ganglion cells: Primary cultures of retinal ganglion cells (RGC) were isolated from retinae of adult Long Evans rat (8-week old) with an immunopanning technique, according the protocol previously described in young rats by Barres et al. (1988). Briefly, animals were anesthetized and killed by cerebral dislocation and their eyes removed and placed in a solution of phosphate-buffered saline (PBS) containing 1 g/1 of glucose (PBS-glucose; Invitrogen, Carlsbad, Calif., USA). After one rinse in PBS-glucose, retinae were incubated in the same medium containing 33 UI/ml of papain (Worthington, Lakewood, N.J., USA) and 200 UI/ml of DNAse (Sigma-Aldrich, St-Louis, MO, USA) for 30 min at 37° C. They were then rinsed in PBS-glucose, containing 0.15% ovomucoid (Roche Diagnosis, Basel, Switzerland) and 0.15% bovine serum albumin (BSA; Sigma-Aldrich). Retina were dissociated in PBS-glucose containing 0.15% ovomucoid, 015% BSA, 333 UI/ml of DNAse and a rabbit anti-rat macrophage (˜5 mg/ml; Accurate Chemical & Scientific Corporation, Westbury, N.Y., USA) in three steps, using pipettes with decreasing tip diameters. The cell suspension was centrifuged at 115 g during 13 min at room temperature. The supernatant was removed and cells were suspended in PBS-glucose, containing 1% ovomucoid and 1% BSA. After a second centrifugation (115 g, 13 min), cells were suspended in the PBS-glucose, containing 0.02% Bovine Serum Albumin (BSA). Cell suspension was filtrated using a Sefar Nitrex mesh (48 μm, Dutscher, Brumath, France) and then incubated in a dish (diameter 150 mm), previously coated with a goat anti rabbit IgG (Jackson Immunoresearch, West Grove, Pa., USA), during 36 min at room temperature. After a vigorous shaking of the dish, the cell suspension was moved into a second dish (diameter 150 mm), previously coated with the same antibody, and incubated during 33 min at room temperature. After another vigorous shaking, the remaining cell suspension was transferred into a dish (diameter 100 mm), previously coated successively with (i) a goat anti-mouse IgM (Jackson Immunoresearch, West Grove, Pa., USA) and (ii) an hybridoma extract prepared in our laboratory from a T11D7 hybridoma cell line (ATCC, Manassas, Va., USA). After 45 min incubation, the dish was rinsed ten times with PSB-glucose. Adherent cells remaining into the dish were RGC specifically selected by Thy-1 antibody contained in the hybridoma extract. Cells were incubated with Earle's Balanced Salts Solution (EBSS; Sigma-Aldrich) containing 0.125% of trypsin (Sigma-Aldrich) for 10 min at 37° C., in humidified atmosphere (5% CO₂). Trypsin action was blocked by addition into dish of PBS-glucose, containing 30% inactive foetal bovine serum (FBS; Invitrogen). Cells were detached by ˜10 successive pipette flows of PBS-glucose-30% FBS, and the resulting cell suspension was centrifuged at 115 g for 15 min. Pure RGC were then suspended in Neurobasal-A medium (Invitrogen) supplemented 2 mM L-glutamine (Invitrogen) and cells were seeded in the 48-well plate at an initial density of 2×10⁴ cells/well, on 8 mm in diameter coverslips, previously coated by successively poly-D-lysine (2 μg/cm² for 45 min; Sigma-Aldrich) and laminin (1 μg/cm² overnight; Sigma-Aldrich). Cultures were kept in a humidified chamber at 37° C. containing 5% CO₂, for 1 to 6 Days in vitro (DIV).

Viability Test and RCG Counting:

RGC viability was assessed with the “lived-dead” test (Invitrogen), which consists in labelling viable cells with calcein AM detected as a green fluorescence, whereas dead cell were labelled with ethidium producing a red fluorescence. Briefly, coverslips were incubated in a mixture of calceinAM and ethidium homodimer-1 (performed in a PBS medium) for 1 hour in the incubator (humidified chamber, 37° C., 5% CO₂). Only lived RCG were counted from seven fields taken on each coverslip using a microscope (Leica DM 5000B, Solms, Germany) equipped for epifluorescence. Viable RCG were counted at 1 day in vitro (DIV) and 6 DIV to calculate the percentage of cell survival.

Results:

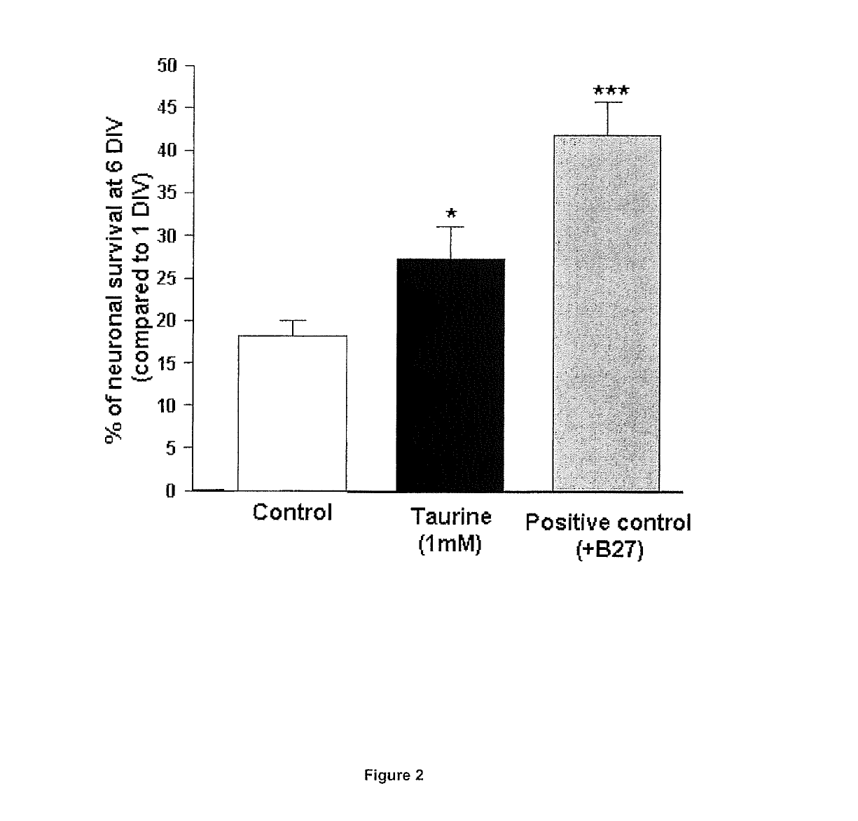

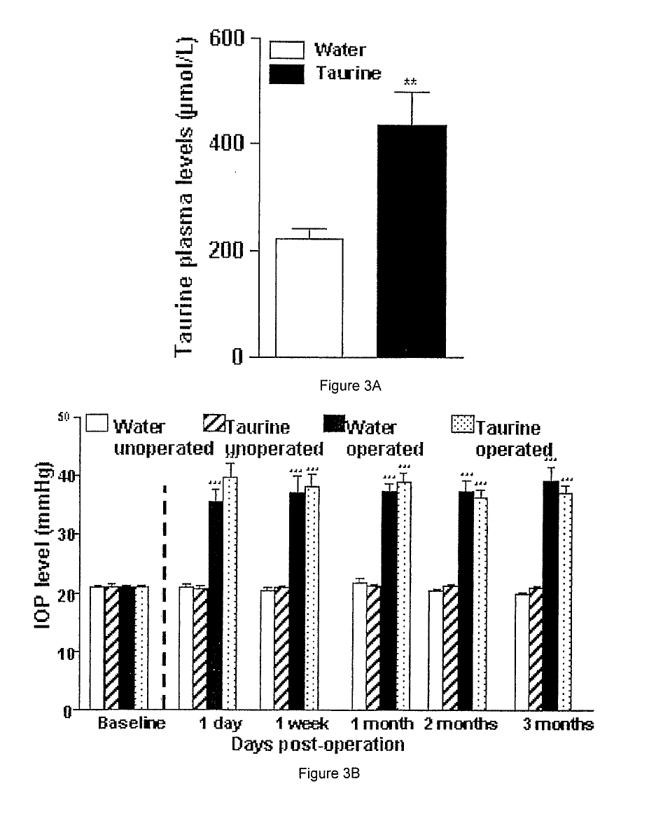

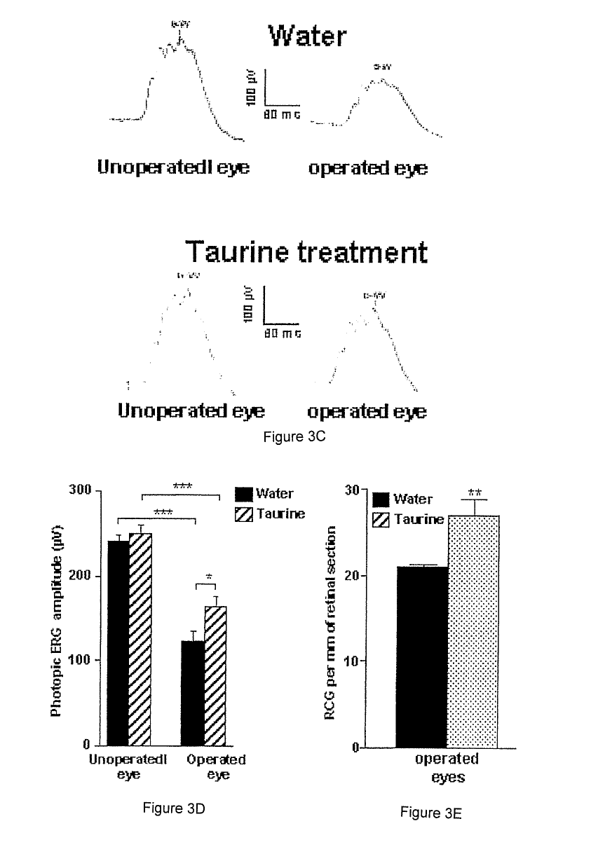

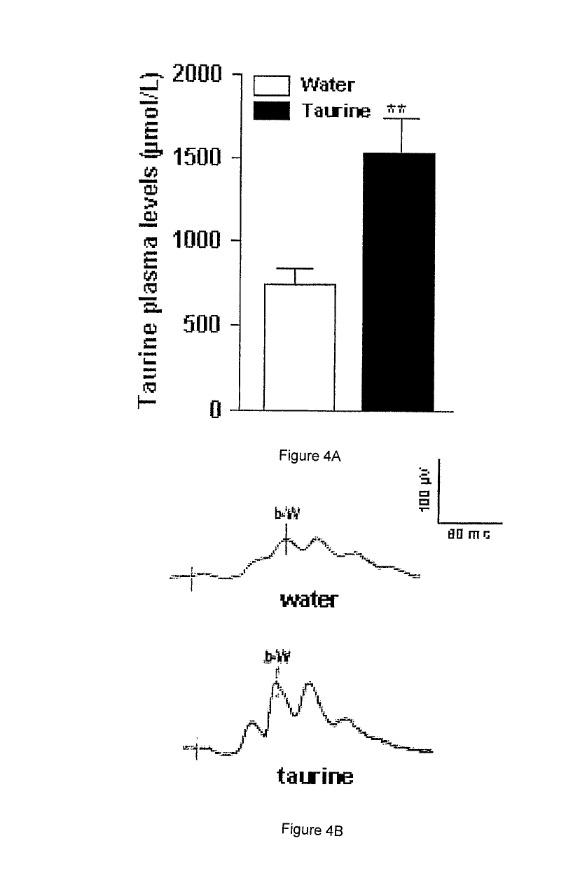

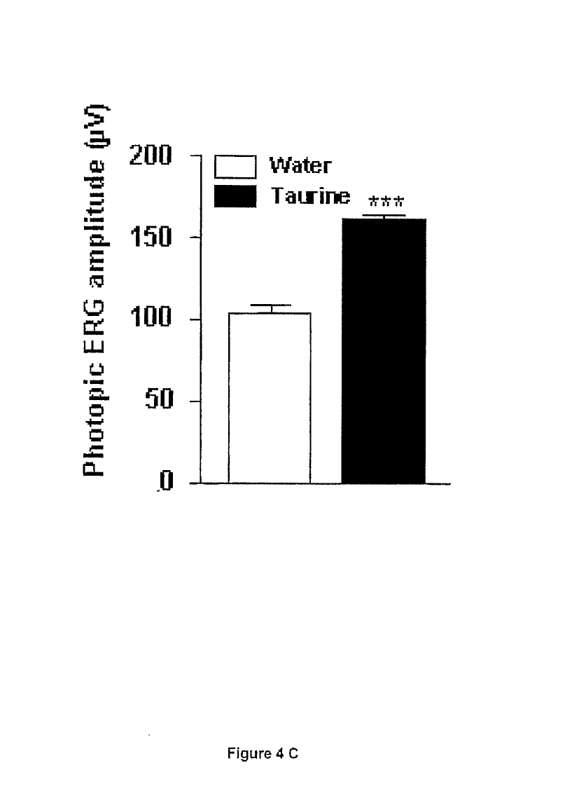

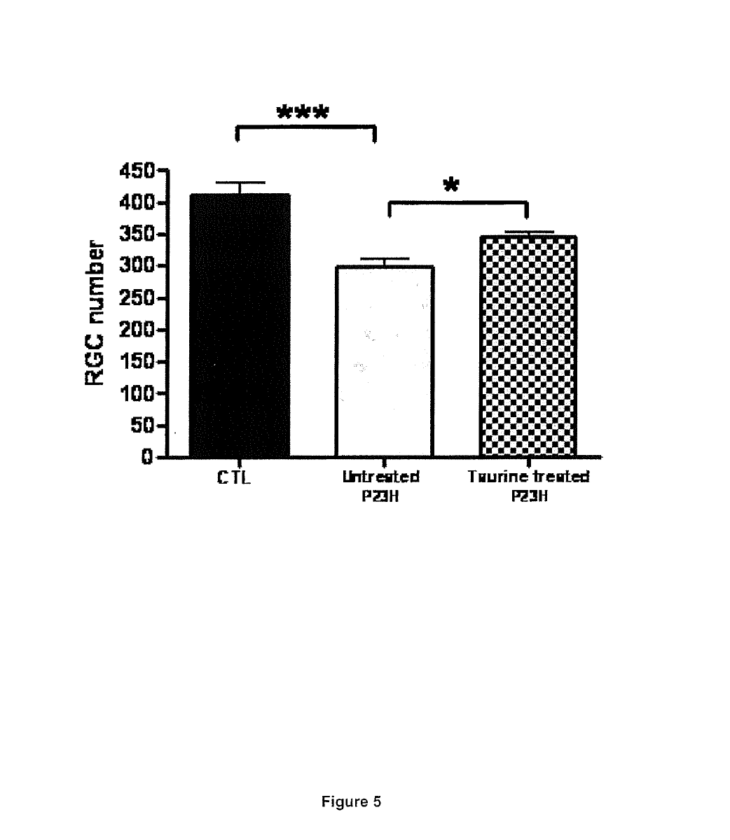

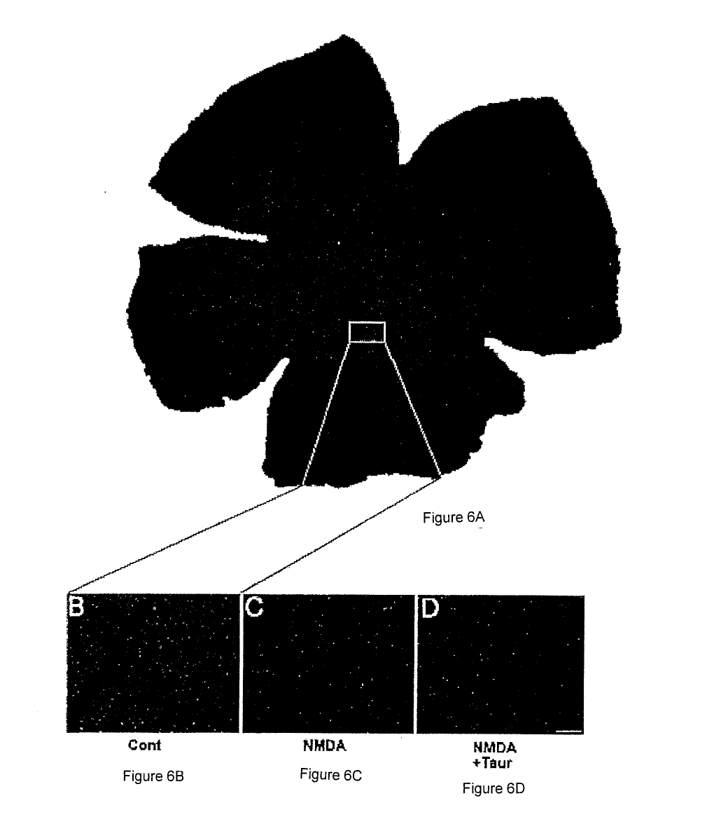

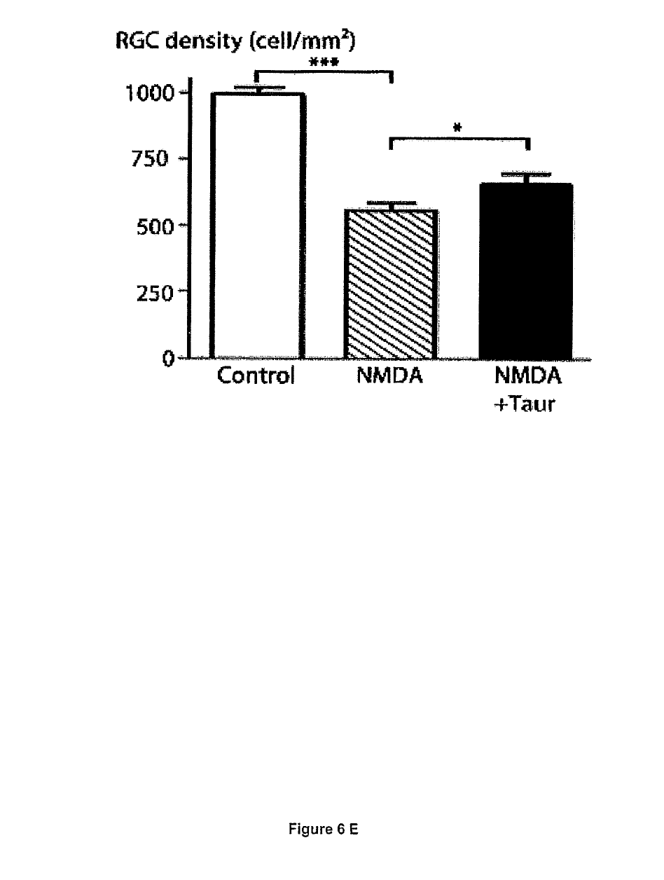

To investigate if taurine has a direct effect on RGC survival, it was applied on pure adult rat RGC in culture. After 6 days in culture, taurine increased the number of surviving cells by 49% from 18.2±1.8% in the control conditions to 27.2±3.7% (FIG. 2). This result indicated that taurine can affect directly RGC survival.

### EXAMPLE 3

### Protective Action of Taurine in Animal Model of Glaucoma or of Pigmentary Glaucoma or of Retinitis Pigmentosa

Material & Methods:

Animal Treatments:

Long-Evans rats (8-week old) were purchased from Janvier (Le Genest-St-Isle, France) whereas DBA/2J mice (8-month old) were purchased from Charles River (Larbresle, France). Heterozygous P23H rats (line 1) and Sprague Dawley rats (n=7) were bred at Charles River (Saint Aubin-les-Elbeuf, France). For taurine supplementation, animals were divided in two groups: a control group and a group receiving taurine in its drinking water. Taurine (0.2 M) was added to the drinking water during 3 months for Long-Evans rats with vascular occlusion and during 4 months to DBA/2J mice. At 9 months, a group of P23H rats (n=6) received taurine supplementation (0.1M) in the drinking water until 12 months while another group (n=7) was maintained on water without added taurine. Animals drinking water without added taurine were always considered as control animals.

Occlusion of Episcleral Veins by Cauterization:

Occlusion of episcleral veins was performed on 8 week-old Long-Evans or Wistar rats as described previously (Mittag et al., 2000). Animals were anesthetized with a mixture Ketamine (100 mg/Kg; Virbac, France) and, Xylazine (10 mg/Kg; Bayer, Leverkusen, Germany), and a local anesthesia (Tetracaine, Laboratoires TVM, Lempdes, France) were administrated on the cornea. At the level of the right eye, three episcleral veins, superonasal, superotemporal and inferotemporal, were emerged by removing the conjunctive tissue. Vein occlusion was realized by cauterization, using Aesculap® bipolar forceps (B. Braun, Melsungen, Germany). The left eye was not operated and considered as a control eye. After surgery, anti-inflammatory ointment (Sterdex, Novartis, Basel, Switherland) was applied on the two eyes.

Intra-Ocular Pressure Measurements:

IOP was measured from right and left eyes on awake animals using a Tonolab tonometer (Icare, Helsinki, Finland). The tonometer was applied perpendicularly to the cornea and three successive measures were recorded and the average was taken for the final value of IOP.

Electroretinogram (ERG) Recording:

Photopic ERGs were performed on cauterized and control eyes from rats and DBA2/J mice. Animals were anesthetized by an intramuscular injection with a solution containing a mixture of Ketamine (100 mg/Kg) and, Xylazine (10 mg/Kg). Pupils were dilated by a topical application of Mydriaticum, Laboratoires Thea, Clermont-Ferrand, France). ERG were recorded using two gold loop electrodes were placed on the corneal surface of each eye and maintained with 3% Ocry-gel. These recording electrodes were respectively referenced with two steel electrodes, inserted sub-cuteanously at the level of each cheek of animals. A needle electrode, also inserted sub-cuteanously, at the level of the basis of the tail served as ground. Responses to light stimulus were amplified, filtered and digitalized using the “Multiliner vision” program.

Histology:

Animals were anesthetized and killed by cerebral dislocation and their eyes were removed and placed into a fixative solution containing 4% paraformaldehyde (Merck chemicals, Darmstadt, Germany) for 24 hours. Eye cups were isolated and cryoprotected in successive solutions of PBS containing 10%, 20% and 30% sucrose at 4° C., oriented along the dorso-ventral axis and embedded in NEG50® (Microm, Francheville, France). Retinal sections were permeabilized for five minutes in PBS containing 0.1% Triton X-100 (Sigma, St. Louis, Mo.), rinsed, and incubated in PBS containing 1% bovine serum albumin (Sigma) and 0.05% Tween 20 (Sigma) for one hour at room temperature. The primary Brn3a monoclonal antibody (1:100; Chemicon or Millipore Corporation, Billerica, Mass., USA) was added to the solution and incubated overnight at 4° C. Sections were rinsed and then incubated with the secondary antibody, rabbit anti-mouse IgG AlexaFluor® 594 (1:500, Invitrogen) for one hour. Cell nuclei were revealed incubating the specimens with 5 uM/ml DAPI in PBS. Sections were rinsed three times with PBS and mounted with Permafluor® reagent (Microm, Francheville, France) and viewed with a Leica DM 5000B microscope (Leica, Solms, Germany) equipped with a Photometrics cool SNAP™ FX camera (Ropper scientific, Evry, France). Quantifications were achieved on whole vertical sections cut along the dorso-ventral axis and crossing the optic nerve.

Statistical Analysis:

Statistical analysis of the results was performed on raw data. An unpaired student t test was used to compare means of 2 groups. For more than 2 groups compared, a one-way ANOVA was used for variance analysis, followed in case of significance by a Bonferroni post-hoc test to compare the means of each group. Differences were considered significant at *P<0.05; **P<0.01; and ***P<0.001.

Results:

In glaucoma, RGC degeneration was related to a decrease in the ocular vascular perfusion (Leske, 2009), which could result in a decrease taurine supply to the retina. To examine whether such a decrease taurine supply could be responsible for this RGC degeneration in glaucoma, we tested if taurine supplementation could prevent the RGC degeneration in two animal models of glaucoma: 1) rats with induced episcleral vein occlusions and 2) DBA/2J mice. Occlusion of episcleral veins by cauterization is indeed a validated model of glaucoma because this operation induced a long-term increase in intra-ocular pressure (FIG. 3B) (see Shareef et al, 1995). In this model, the taurine treatment increased the taurine plasma levels (FIG. 3A). However, it did not affect the increase in IOP level induced by the vein occlusion (FIG. 3B). In this model, ERG recordings revealed a decrease in amplitude on the operated eye as compared to the unoperated eyes in control (water) animals. When animal received taurine supplementation (0.2M in drinking water), a similar decrease in photopic ERG amplitudes was observed, however, this decrease was less important than in animals without taurine supplementation (FIGS. 3C and 3D). The partial prevention of the ERG decrease with the taurine supplementation was statistically significant with respect to that of the operated eye of animals without taurine supplementation (Water) (FIG. 3D). To further examine whether this functional measurement was reflecting an increase in RGC survival, RGC were counted on retinal sections following the cell immunolabelling with a Brn-3a antibody, which labels more than 92% of RGCs (Nadal-Nicolas et al., 2009). This quantification showed a greater number of Brn-3a immunopositive RGC in retinal sections of operated eyes from taurine-treated animals in comparison to those of operated eyes from animals drinking water without taurine supplementation (FIG. 3E). These results indicated that taurine supplementation preserved RGC in the glaucoma animal model generated by episcleral vein occlusion.

To further assess the taurine influence on RGC degeneration, we used the DBA/2J mice, a model for pigmentary glaucoma (see John et al, 1998). In these mice, taurine supplementation from 8 to 12 months induced a significant increase in taurine plasma level (FIG. 4A). Again, the taurine supplementation partially prevented the decrease in photopic ERG amplitudes when compared to control DBA/2J mice drinking pure water (FIGS. 4B and 4C). These results were in agreement with a functional rescue of RGC in this other model of glaucoma.

In retinal dystrophies, RGC degeneration is occurring in human patients (Humayun et al., 1999) as well as in animal models (Wang et al., 2000; Jones et al., 2003). This RGC degeneration is occurring in parallel to a blood vessel atrophy (Penn et al., 2000; Wang et al., 2000). Therefore, to determine if the blood vessel atrophy and the resulting decrease in taurine supply could be responsible for the RGC degeneration, we investigated if RGC degeneration described in the P23H rat (Jones et al., 2003), an animal model of retinal dystrophy and more precisely of retinitis pigmentosa, could be prevented by taurine supplementation in heterozygous line 1 P23H rats (Kolomiets et al., 2010). To determine the extent of RGC degeneration in homozygous P23H rats, RGC nuclei were immunolabelled with the Brn3a antibody. At 1 year, the number of RGCs was 27.4% lower in heterozygous P23H animals (line 1) than in control Sprague Dawley rats. This difference is statistically significant (P<0.001, unpaired t-test). When taurine supplementation (0.1 M) was added to the drinking water, 40% of this RGC loss was prevented (FIG. 5). One way ANOVA followed by Newman-Keuls Multiple Comparison Test showed that the difference between the number of RGCs in heterozygous P23H rats with or without taurine supplementation was statistically significant (P<0.01). This result indicated that taurine supplementation can prevent RGC loss in retinal dystrophies with photoreceptor degeneration (retinitis pigmentosa, Usher diseases, Stargardt diseases, Leber congenital amaurosis). such a RGC protection could be very important for all rehabilitative strategies like retinal prostheses (Zrenner et al., 1999) and optogenetic therapies (Lagali et al., 2008).

The implication of glutamate excitotoxicity in glaucoma was previously demonstrated in different models of glaucoma; memantine, an antagonist at glutamate N-methyl-D-aspartate (NMDA) receptors, was even shown to exert an RGC neuroprotection in these models [Seki M et al., 2008]. To explore if taurine could also protect RGCs from glutamate excitotoxicity, rat retinal explants were incubated in the presence of the glutamate receptor agonist NMDA (100 μM) for 4 days with or without taurine (1 mM). At the end of the incubation, RGCs were immunolabeled with an anti-Brn-3a antibody. When RGCs were counted on the whole flat-mounted retinal explants with an automated platform (see FIG. 6A), we found that NMDA induced a drastic reduction (−44%) in RGC density (Control: 996.0±26.5 cells/mm2, n=33; NMDA: 555.6±31.8 cells/mm2, mean±s.e.m., n=31; p<0.001; FIG. 6B-C, 6E). The co-incubation of taurine with NMDA rescued a significant part of the cell loss (+22%; NMDA+Taur: 655.7±44.4 cells/mm2, mean±s.e.m., n=23, p<0.05, FIG. 6D, 6E), showing thereby that taurine can partially prevent the RGC loss from glutamate excitotoxicity.

### CONCLUSIONS

These results indicated that taurine supplementation can be considered as general treatment to prevent RGC degeneration because it is efficient when taurine plasma concentration is decreased (drug toxicity, vigabatrin), when ocular vascular perfusion is reduced (glaucoma, diabetic retinopathy) or finally following blood vessel atrophy (retinal dystrophies).

### REFERENCES

Aicardi J, Mumford J P, Dumas C, Wood S (1996) Vigabatrin as initial therapy for infantile spasms: a European retrospective survey. Sabril IS Investigator and Peer Review Groups. Epilepsia 37:638-642.
Andersen L, Sundman L O, Lindén I B, Kontro P, Oja S S (1984) Synthesis and anticonvulsant properties of some 2-aminoethanesulfonic acid (taurine) derivatives. J Pharm Sci 73: 106-108.
Buncic J R, Westall C A, Panton C M, Munn J R, MacKeen L D, Logan W J (2004) Characteristic retinal atrophy with secondary “inverse” optic atrophy identifies vigabatrin toxicity in children. Ophthalmology 111:1935-1942.
Chiron C, Dumas C, Jambaque I, Mumford J, Dulac O (1997) Randomized trial comparing vigabatrin and hydrocortisone in infantile spasms due to tuberous sclerosis. Epilepsy Res 26:389-395.
Frisen L, Malmgren K (2003) Characterization of vigabatrin-associated optic atrophy. Acta Ophthalmol Scand 81:466-473. Humayun M S, Prince M, de Juan E, Jr., Barron Y, Moskowitz M, Klock I B, Milam A H (1999) Morphometric analysis of the extramacular retina from postmortem eyes with retinitis pigmentosa. Invest Ophthalmol Vis Sci 40:143-148.
Jones B W, Watt C B, Frederick J M, Baehr W, Chen C K, Levine E M, Milam A H, Lavail M M, Marc R E (2003) Retinal remodeling triggered by photoreceptor degenerations. J Comp Neurol 464:1-16.
John S W, Smith R S, Savinova O V, Hawes N L, Chang B, Turbull D, Davisson M, Roderick T H, Heckenlively J R (1998) Essential iris atrophy, pigment dispersion, and glaucoma in DBA/2J mice. Invest Ophtalmol Vis Sci 39: 951-962.
Kolomiets B, Dubus E, Simonutti M, Rosolen S, Sahel J A, Picaud S (2010) Late histological changes in the P23H rat retina after photoreceptor loss. Neurobiol Dis In press.
Kontro P, Lindén IB, Gothóni G, Oja S S. (1983) Novel anticonvulsivant taurine derivates. Prog Clin Biol Res, Vol. 125: 211-220)
Lagali P S, Balya D, Awatramani G B, Munch T A, Kim D S, Busskamp V, Cepko C L, Roska B (2008) Light-activated channels targeted to ON bipolar cells restore visual function in retinal degeneration. Nat Neurosci 11:667-675.
Leske M C (2009) Ocular perfusion pressure and glaucoma: clinical trial and epidemiologic findings. Curr Opin Ophthalmol 20:73-78.
Libby, R. T., D. B. Gould, M. G. Anderson, and S. W. John. (2005). Complex genetics of glaucoma susceptibility. Annu. Rev. Genomics Hum. Genet. 6:15-44.
Lux A L, Edwards S W, Hancock E, Johnson A L, Kennedy C R, Newton R W, O'Callaghan F J, Verity C M, Osborne J P (2004) The United Kingdom Infantile Spasms Study comparing vigabatrin with prednisolone or tetracosactide at 14 days: a multicentre, randomised controlled trial. Lancet 364:1773-1778. Mittag T W, Danias J, Pohorenec G, Yuan H M, Burakgazi E, Chalmers-Redman R, Podos S M, Tatton W G (2000) Retinal damage after 3 to 4 months of elevated intraocular pressure in a rat glaucoma model. Invest Ophthalmol Vis Sci 41:3451-3459.
Mittag T W, Danias J, Pohorenec G, Yuan H M, Burakgazi E, Chalmers-Redman R, Podos S M, Tatton W G (2000) Retinal damage after 3 to 4 months of elevated intraocular
Nadal-Nicolas F M , Jimenez-Lopez M, Sobrado-Calvo P, Nieto-Lopez L, Canovas-Martinez I, Salinas-Navarro M, Vidal-Sanz M, Agudo M (2009) Brn3a as a marker of retinal ganglion cells: qualitative and quantitative time course studies in naive and optic nerve-injured retinas. Invest Ophthalmol Vis Sci 50:3860-3868.
Penn J S, Li S, Naash M I (2000) Ambient hypoxia reverses retinal vascular attenuation in a transgenic mouse model of autosomal dominant retinitis pigmentosa. Invest Ophthalmol Vis Sci 41:4007-4013.
Quigley, H. A. (1987). Reappraisal of the mechanisms of glaucomatous optic nerve damage. Eye. 1:318-322.
Quigley, H. A. (1999). Neuronal death in glaucoma. Prog. Retin. Eye Res. 18:39-57.
Seki M & Lipton S A (2008) Targeting excitotoxic/free radical signaling pathways for therapeutic intervention in glaucoma. Progress in brain research 173:495-510.
Shareef S R, Garcia-Valenzuela E, Salierno A, Walsh J, Sharma S C (1995) Chronic ocular hypertension following episcleral venous occlusion in rats. Exp Eye Res 61: 379-382.
Viestenz A, Viestenz A, Mardin C Y (2003) [Vigabatrin-associated bilateral simple optic nerve atrophy with visual field constriction. A case report and a survey of the literature]. Ophthalmologe 100:402-405. Wang S, Villegas-Perez M P, Vidal-Sanz M, Lund R D (2000) Progressive optic axon dystrophy and vacuslar changes in rd mice. Invest Ophthalmol Vis Sci 41:537-545.
Wang S, Villegas-Perez M P, Vidal-Sanz M, Lund R D (2000) Progressive optic axon dystrophy and vacuslar changes in rd mice. Invest Ophthalmol Vis Sci 41:537-545.
Whitmore, A. V., R. T. Libby, and S. W. M. John. 2005. Glaucoma: thinking in new ways—a role for autonomous axonal self-destruction and other compartmentalised processes? Prog. Retin. Eye Res. 24:639-662. Zrenner E, Stett A, Weiss S, Aramant R B, Guenther E, Kohler K, Miliczek K D, Seiler M J, Haemmerle H (1999) Can subretinal microphotodiodes successfully replace degenerated photoreceptors? Vision Res 39:2555-2567.
Zrenner E, Stett A, Weiss S, Aramant R B, Guenther E, Kohler K, Miliczek K D, Seiler M J, Haemmerle H (1999) Can subretinal microphotodiodes successfully replace degenerated photoreceptors? Vision Res 39:2555-2567.

1. A method for the prevention and treatment of a disease associated with retinal ganglion cell degeneration in a patient in need thereof, comprising the step of
administering to the patient a substance selected from the group consisting of taurine, a taurine precursor, a taurine metabolite, a taurine derivative, a taurine analog and a substance required for taurine biosynthesis.

2. The method of claim 1, wherein the disease associated with retinal ganglion cell degeneration is selected from the group consisting of diseases associated with optic nerve atrophy and pathologies associated with retinal ischemia.

3. The method of claim 1, wherein the disease associated with retinal ganglion cell degeneration is selected from the group consisting of arteritic ischemic optic neuropathy, arteritic ischemic optic neuropathy associated with giant cell arteritis, nonarteritic ischemic optic neuropathy, infiltrative optic neuropathy, infiltrative optic neuropathy associated with sarcoidosis, infectious optic neuropathy, infectious optic neuropathy associated with syphilis, infectious optic neuropathy associated with Lyme disease, infectious optic neuropathy associated with toxoplasmosis, infectious optic neuropathy associated with herpes zoster, optic neuritis from demyelinating disease, posradiation optic neuropathy, acrodermatitis enteropathica, hereditary optic neuropathy, hereditary optic neuropathy associated with dominant optic neuropathy, compressive optic neuropathy, compressive optic neuropathy associated with orbital pseudotumor, compressive optic neuropathy associated with thyroid eye disease, autoimmune optic neuropathy, and autoimmune optic neuropathy associated with Lupus.

4. The method of claim 1, wherein said taurine precursor is selected from the group consisting of cysteine, cystathionine, homocysteine, S-adenosylhomocysteine, serine, N-acetyl-cysteine, glutathione, N-formylmethionine, S-adenosylmethionine, betaine and methionine.

5. The method of claim 1, wherein said taurine metabolite is selected from the group consisting of hypotaurine, thiotaurine, taurocholate, and tauret (retinyliden taurine).

6. The method of claim 1, wherein said taurine derivative is selected from the group consisting of acetylhomotaurinate, and piperidino-, benzamido-, phthalimido- or phenylsuccinylimido taurine derivatives taurolidine, taurultam and taurinamide, chlorohydrate-N-isopropylamide-2-(1-phenylethyl)aminoethanesulfonic acid.

7. The method of claim 1, wherein said taurine analog is selected from the group consisting of (+/−)piperidine-3-sulfonic acid (PSA), 2-aminoethylphosphonic acid (AEP), (+/−)2-acetylaminocyclohexane sulfonic acid (ATAHS), 2-aminobenzenesulfonate (ANSA), hypotaurine, ±trans-2-aminocyclopentanesulfonic acid (TAPS) 8-tetrahydroquinoline sulfonic acid (THQS), N-2-hydroxyethylpiperazine-N′-2-ethane sulphonic acid (HEPES), beta-alanine, glycine, guanidinoethylsulfate (GES), and 3-acétamido-1-propanesulfonic acid (acamprosate).

8. The method of claim 1, wherein the substance required for the taurine biosynthesis is selected from the group consisting of vitamin B6, vitamin B12, folic acid, riboflavin, pyridoxine, niacin, thiamine, and pantothenic acid.

9. A pharmaceutical composition for the prevention and treatment of a disease associated with retinal ganglion cell degeneration which comprises a selected from the group consisting of taurine, a taurine precursor, a taurine metabolite, a taurine derivative, a taurine analog and a substance required for taurine biosynthesis and at least one active ingredient selected from the groups consisting of A-D:
A. latanoprost, timolol, travoprost, dorzolamide, brimonidine, bimatoprost, apraclonidine, dipivephrine, propine, acetazomide, and brinzolamide;
B. chloramphenicol, chloroquine, clioquinol, dapsone, ethambutol, iodochlorohydroxyquinoline, isoniazide, linezolid, and streptomycin;
C. cyclosporine, interferon-alpha, and tacrolimus (FK506);
D. carboplatin, chlorambucil, cisplatin, 5-fluorouracil, methotrexate, a nitrosurea, paclitaxel, tamoxifen, 5-vincristine, cytosine arabinoside, purine analogues, procarbazine, cyclophosphamide, and vinca alkaloids; and
E. at least one active ingredient selected from the group consisting of amiodarone, amantidine amoproxen, cafergot, chlorpropamide, cimetidine, clomiphene citrate, deferoxamine, disulfiram, emetine, infliximab, pheniprazine, quinine, a phosphodiesterase (PDE) inhibitor, bendroflumethiazide, chorothiazide, chlortalidone, hydrochlorothiazide, hydroflumethiazide, indapamide, methyclothiazide, metolazone, polythiazide, and trichlormethiazide.

10. The method of claim 2, wherein the disease associated with optic nerve atrophy is selected from the group consisting of glaucoma and Leber hereditary optic neuropathy.

11. The method of claim 2, wherein the pathology associated with retinal ischemia disease is a vascular occlusion.

12. The pharmaceutical composition of claim 9, further comprising one or more pharmaceutically acceptable excipients.

13. The chemotherapeutic composition of claim 9, wherein the nitrosurea is selected from the group consisting of bis-chloroethylnitrosourea (BCNU), N-(2-chloroethyl)-N-cyclohexyl-N-nitrosourea (CCNU), and N′-[(4-amino-2-methylpyrimidin-5-yl)methyl]-N-(2-chloroethyl)-N-nitrosourea (ACNU).

14. The therapeutic composition of claim 9 wherein the PDE inhibitor is selected from the group consisting of sildenafil, tadalafil, and vardenafil.

15. A method for the prevention and/or treatment of glaucoma in a patient in need thereof, comprising the step of
administering to the patient a therapeutically effective amount of a substance selected from the group consisting of taurine, a taurine precursor, a taurine metabolite, a taurine derivative, a taurine analog and a substance required for taurine biosynthesis.
